(19) United States
(12) Patent Application Publication
(10) Pub. No.: US 2024/0145079 A1
(43) Pub. Date: May 2, 2024

(54) PRESENTING BIOSENSING DATA IN CONTEXT

(71) Applicant: Microsoft Technology Licensing, LLC, Redmond, WA (US)

(72) Inventors: Aashish PATEL, San Diego, CA (US); Hayden HELM, San Francisco, WA (US); Jen-Tse DONG, Bellevue, WA (US); Siddharth SIDDHARTH, Redmond, WA (US); Weiwei YANG, Seattle, WA (US); Amber D. HOAK, Silverdale, WA (US); David A. TITTSWORTH, Gig Harbor, WA (US); Kateryna LYTVYNETS, Redmond, WA (US)

(73) Assignee: Microsoft Technology Licensing, LLC, Redmond, WA (US)

(21) Appl. No.: 17/977,672

(22) Filed: Oct. 31, 2022

(51) Int. Cl. G16H 40/63

(52) U.S. Cl. CPC G16H 40/63

(57) ABSTRACT

Biosensing measurements (e.g., heart rate, pupil size, cognitive load, stress level, etc.) are communicated in the context of events that occurred concurrently with the biosensing measurements. The biosensing measurements and the contextual events can be presented in real-time or as historical summaries. Such presentations allow users to easily gain useful insights into which specific events triggered which specific physiological responses in users. Therefore, the present concepts more effectively communicate insights that can be used to change user behavior, modify workflow, design improved products or services, enhance user satisfaction and wellbeing, increase productivity and revenue, and eliminate negative impacts on user's emotions and mental state.

### BACKGROUND

Neuroergonomics is a field of study that applies the principles of neuroscience (the study of the nervous system using physiology, biology, anatomy, chemistry, etc.) to ergonomics (the application of psychology and physiology to engineering products). For example, neuroergonomics includes studying the human body, including the brain, to assess and improve physical and cognitive conditions. The potential benefits of neuroergonomics include increased productivity, better physical and mental health, and improved technological designs.

### SUMMARY

The present concepts involve presenting biosensing data in context such that useful knowledge, including neuroergonomic insights, can be gained. For instance, biosensing measurements associated with one or more users can be presented in conjunction with events that occurred when the biosensing measurements were taken, so that the viewer can observe how the users responded to the events. Current biosensing measurements can be presented in real-time as the live events are occurring. Furthermore, historical biosensing measurements can be presented, in summary format, in synchronization with past events.

Biosensing data can include one or multiple modes of sensor data that includes biological, physiological, and/or neurological signals from the body as well as environmental sensors and digital applications. Biosensing data can also include cognitive states data, which can be inferred from the sensor data using machine learning models. The context in which the biosensing data are measured can be any scenario, such as videoconference meetings, entertainment shows, speeches, news articles, games, advertisements, etc. The timing of the events occurring in the context is aligned with the timing of the biosensing data.

By presenting biosensing measurements in synchronization with specific events, greater insights can be observed from the presentation. First, neuroergonomic responses will make more sense to the viewer because the neuroergonomic responses are presented in conjunction with the events that triggered those responses. Second, positive and negative neuroergonomic responses can provide feedback about positive and negative aspects of the context, such as whether certain words in a speech trigger negative emotions among the audience, whether an advertisement generated positive cognitive states among the target viewers, whether a workplace task resulted in high stress for an employee, etc. The feedback can be used to effect changes (such as changing user behavior or changing a product) that reduce or avoid negative responses and instead promote positive responses instead.

### BRIEF DESCRIPTION OF THE DRAWINGS

The detailed description below references accompanying figures. The use of similar reference numbers in different instances in the description and the figures may indicate similar or identical items. The example figures are not necessarily to scale. The number of any particular element in the figures is for illustration purposes and is not limiting.

FIG. 1 illustrates an example use of sensors, consistent with some implementations of the present concepts.

FIG. 2 illustrates an example neuroergonomic system, consistent with some implementations of the present concepts.

FIG. 3 illustrates an example live presentation, consistent with some implementations of the present concepts.

FIG. 4 illustrates an example historical presentation, consistent with some implementations of the present concepts.

FIG. 5 illustrates an example context display primitive, consistent with some implementations of the present concepts.

FIGS. 6A-6D illustrate example heart rate display primitives, consistent with some implementations of the present concepts.

FIGS. 7A-7C illustrate example cognitive state display primitives, consistent with some implementations of the present concepts.

FIGS. 8A and 8B illustrate example cognitive load display primitives, consistent with some implementations of the present concepts.

FIGS. 9A and 9B illustrate example electroencephalogram (EEG) display primitives, consistent with some implementations of the present concepts.

FIG. 10 illustrates a flowchart of an example neuroergonomic method, consistent with some implementations of the present concepts.

FIG. 11 illustrates example configurations of a neuroergonomic system, consistent with some implementations of the present concepts.

### DETAILED DESCRIPTION

### Technical Problems

The availability of sensors have been increasing. Cameras are ubiquitously found on many types of devices (e.g., smartphones, laptops, vehicles, etc.). Heart rate monitors, which used to be available only in hospitals, are now found in gymnasiums (e.g., on treadmill handlebars) and on wearables (e.g., smartwatches). And, consumer-grade brain-computer interface (BCI) devices, such as EEG sensors for home use, are on the rise. Sensor data from these sensors can be presented to a user in a myriad of formats, such as numerical values, graph line charts, bar graphs, etc.

Furthermore, some sensor data can be used to infer cognitive states (e.g., cognitive load, affective state, stress, and attention) of users by machine learning models that are trained to predict the cognitive states based on sensor data. Recent advances in artificial intelligence computing and the availability of large datasets of sensor readings for training have enabled the development of fast and accurate prediction models. Similar to the sensor data, the cognitive state data and other physiological information can also be presented to a user in many types of graphical formats.

However, simply outputting biosensing data (such as the sensor data and the cognitive state data) to a user is ineffective in communicating useful insights other than the data itself. For example, a conventional consumer-facing electrocardiogram (EKG) monitor displays the user's heart rate but does not provide any contextual information that would convey what event or stimulus caused the user's heart rate to rise or fall. A historical graph of a user's body temperature taken by a smartwatch throughout the day does not explain why the user's body temperature increased or decreased at various times, because the graph is not correlated with any contextual events that triggered the changes in body temperature. Similarly, a trend graph of the user's EEG spectral band power measurements alone provides no context as to why certain bands were prominent during specific times. Therefore, there is a need to improve the user experience by presenting biosensing data (e.g., sensor readings and cognitive states) in context of what, who, when, where, why, and how those changes came about.

### Technical Solutions and Advantages

The present concepts involve effectively communicating biosensing measurements along with contextual background information to provide greater insights. Furthermore, integrating the biosensing data, contextual information, and related controls into application products and into graphical user interfaces will improve user experience.

In some implementations of the present concepts, backend services provide biosensing measurements (such as sensor readings and cognitive states) and contextual information (such as background events and external stimuli that occurred concurrently with the biosensing measurements) via application programming interface (API) services. Frontend services or frontend applications generate presentations that communicate biosensing measurements and the contextual information in insightful ways that correlate them to each other.

The presentations can include a set of display primitives (e.g., graphical user interface (GUI) elements) that are tailored to effectively presenting biosensing measurements in conjunction with contextual information. The display primitives can be seamlessly integrated into existing applications such that the neuroergonomic insights can be presented along with other application-related GUI elements.

For example, a conventional EKG monitor displays heart-related measurements, but does not present any context such that an observer can draw valuable insights as to why the heart-related measurements are what they are or why they changed. The present concepts, however, can display heart-related measurements (or other sensor readings and cognitive states) along with contextual data. For example, the heart rate of a video game player can be presented along with contextual data about what events occurred during the video game session that coincided with the heart-related measurements. Therefore, the observer can draw richer insights, for example, that the player's heart rate jumped when the player became excited from almost winning in the video game or that the player's heart rate was low and steady when the player became bored from lack of activity for an extended period of time. Presenting biosensing measurements in alignment with specific contextual event-based information communicates the biosensing measurements to users more effectively than simply presenting the biosensing measurements in the abstract or in isolation without any context.

Furthermore, the present concepts not only enriches the communication of biosensing measurements with contextual data but also provide valuable insights into the quality and effectiveness of products and services that constitute the context under which the biosensing measurements were obtained. For example, the biosensing data taken during a videoconference meeting, during a speech, while an advertisement is being presented, or while playing a movie can provide insights into how to improve the meeting, the speech, the advertisement, or the movie, respectively. The present concepts would be able to highlight whether any part of the videoconference meeting caused stress on the participants, which part of the speech triggered positive or negative emotions from the audience, how the conversation dynamically affected the overall emotional responses of the audience, whether an advertisement successfully captured the consumers' attention, and whether any part of the movie caused the viewers to be bored, and so on. Such insights can be used to improve products and services by enhancing any positive aspects and/or eliminating any negative aspects that are identified by the concurrent biosensing measurements.

Moreover, in addition to conveying useful information, the present concepts can involve an adaptive digital environment that automatically changes (or recommends changes) in real time based on the biosensing data. For example, the frontend application can recommend a break if participants in a videoconference meeting are experiencing fatigue and high stress levels, as indicated by the biosensing data. With the user's permission, such intervening actions can be automatically implemented to benefit the user or can be suggested to the user for approval before implementation.

### Sensors

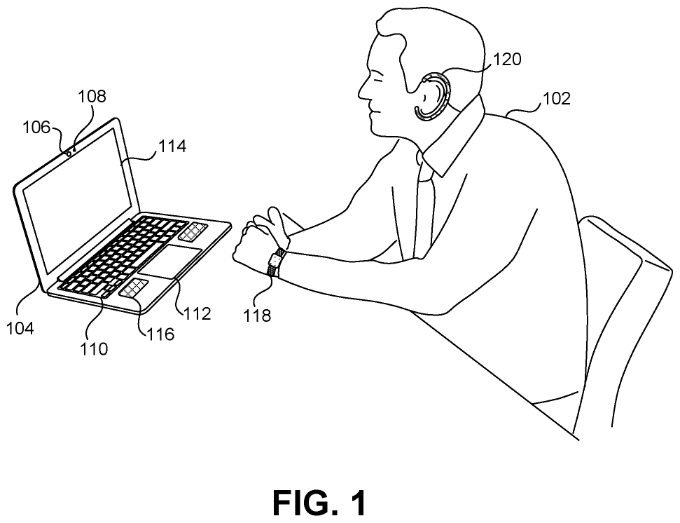

FIG. 1 illustrates an example use of sensors, consistent with some implementations of the present concepts. In this example, a user 102 is using a laptop 104. The user 102 can use the laptop 104 for a myriad of purposes, such as participating in a videoconference meeting with others, playing a video game (a single player game or a multiplayer game with other online players), drafting an email, watching movies, shopping online, etc. The user 102 can choose to opt in and have one or more sensors detect and measure a certain set of inputs associated with the user 102. The inputs can include physiological inputs that take measurements from the user's body, environmental inputs that take measurements from the user's surroundings, and/or digital inputs take measurements from electronics.

For example, the laptop 104 includes a camera 106. The camera 106 can sense the ambient light in the user's environment. The camera 106 can be an infrared camera that measures the user's body temperature. The camera 106 can be a red-green-blue (RGB) camera that functions in conjunction with an image recognition module for eye gaze tracking, measuring pupil dilation, recognizing facial expressions, or detecting skin flushing or blushing. The camera 106 can also measure the user's heart rate and/or respiration rate, as well as detect perspiration.

The laptop 104 also includes a microphone 108 for capturing audio. The microphone 108 can detect ambient sounds as well as the user's speech. The microphone 108 can function in conjunction with a speech recognition module or an audio processing module to detect the words spoken, the user's vocal tone, speech volume, the source of background sounds, the genre of music playing in the background, etc.

The laptop 104 also includes a keyboard 110 and a touchpad 112. The keyboard 110 and/or the touchpad 112 can include a finger pulse heart rate monitor. The keyboard 110 and/or the touchpad 112, in conjunctions with the laptop's operating system (OS) and/or applications, can detect usage telemetry, such as typing rate, clicking rate, scrolling/swiping rate, browsing speed, etc., and also detect the digital focus of the user 102 (e.g., reading, watching, listening, composing, etc.). The OS and/or the applications in the laptop 104 can provide additional digital inputs, such as the number of concurrently running applications, processor usage, network usage, network latency, memory usage, disk read and write speeds, etc.

The user 102 can wear a smartwatch 118 or any other wearable devices, and permit certain readings to be taken. The smartwatch 118 can measure the user's heart rate, heart rate variability (HRV), perspiration rate (e.g., via a photoplethysmography (PPG) sensor), blood pressure, body temperature, body fat, blood sugar, etc. The smartwatch 118 can include an inertial measurement unit (IMU) that measures the user's motions and physical activities, such as being asleep, sitting, walking, running, and jumping.

The user 102 can choose to wear an EEG sensor 120. Depending on the type, the EEG sensor 120 may be worn around the scalp, behind the ear (as shown in FIG. 1), or inside the ear. The EEG sensor 120 includes sensors, such as electrodes, that measure electrical activities of the user's brain.

The above descriptions in connection with FIG. 1 provide a number of example sensors that can measure physiological, environmental, cognitive, and/or digital inputs associated with the user 102. Other types of sensors and other modalities of inputs are possible.

The example sensors described above output sensor data, such as the measurements taken. The sensor data can include metadata, such as timestamps for each of the measurements as well as the identity of the user 102 associated with the measurements. The timestamps can provide a timeline of sensor measurements, such as heart rate trends or body temperature trends over time.

The laptop 104 also includes a display 114 for showing graphical presentations to the user 102. The laptop 104 also includes a speaker 116 for outputting audio to the user 102. The display 114 and/or the speaker 116 can be used to output biosensing data and contextual information to the user 102, as will be explained below.

### System

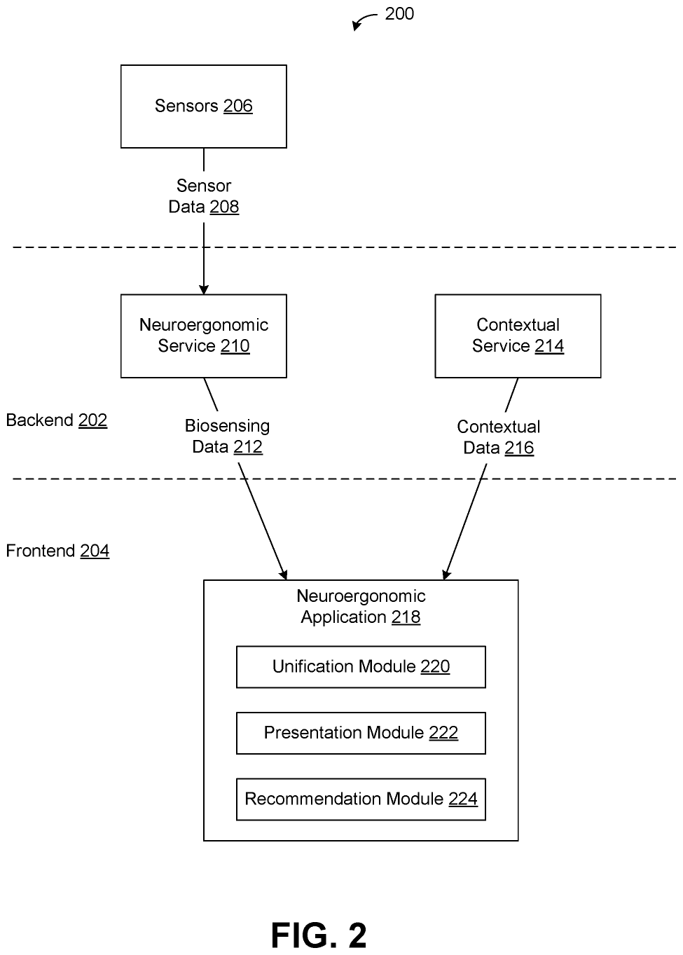

FIG. 2 illustrates an example neuroergonomic system 200, consistent with some implementations of the present concepts. The neuroergonomic system 200 can function as a physiological information service that helps users better understand key physical and cognitive indicators. In one implementation, the neuroergonomic system 200 includes a collection of services and/or applications that are linked together to collect, analyze, calculate, generate, and display biosensing measurements and contextual information. The neuroergonomic system 200 includes a backend 202 and a frontend 204 of hardware devices, software applications, and/or services.

In the example implementation illustrated in FIG. 2, the neuroergonomic system 200 includes sensors 206. The sensors 206 include hardware sensors and/or software sensors that can detect and measure physiological inputs, environmental inputs, and/or digital inputs associated with users. For example, the sensors 206 can include the sensors described above in connection with FIG. 1 for sensing multiple modalities of inputs. The sensors 206 can include contact sensors that attach to the bodies of users or contactless sensors that are proximate to the users.

The sensors 206 output sensor data 208. The sensor data 208 includes measurements taken by the sensor 206. The sensor data 208 can include metadata, such as identities of users associated with the sensor data 208, timestamps that indicate when the sensor data 208 was measured, location data, device identifiers, session identifiers, etc. The timestamps can be used for form a timeline of the sensor data 208, for example, a trend graph line of the sensor measurements over time.

The backend 202 of the neuroergonomic system 200 includes a neuroergonomic service 210. The neuroergonomic service 210 takes in the sensor data 208 and outputs biosensing data 212. That is, if the users opt in and grants permission, the sensor data 208 is used by the neuroergonomic service 210 to infer cognitive states and other physiological states of the users. The neuroergonomic service 210 includes machine learning models that can estimate cognitive states of users based on the sensor data 208. U.S. patent application Ser. No. 17/944,022 (attorney docket no. 412051-US-NP), entitled “Neuroergonomic API Service for Software Applications,” filed on Sep. 13, 2022, describes example artificial intelligence techniques for training and using machine learning models to predict cognitive states of users based on multimodal sensor inputs. The entirety of the '022 application is incorporated by reference herein.

Cognitive states inferred by the neuroergonomic service 210 can include, for example, cognitive load, affective state, stress, and attention. Cognitive load indicates a user's mental effort expended (or the amount of mental resources needed to perform a task) and thus indicate how busy the user's mind is. For example, the user's mind may be fatigued from overusing her mental working memory resources, particularly from long-term mental overload. The affective state indicates whether the user's level of arousal is high or low and indicates whether the user's valence is positive or negative. For example, high arousal and negative valence means that the user is anxious, fearful, or angry. High arousal and positive valence means that the user is happy, interested, joyful, playful, active, excited, or alert. Low arousal and negative valence means that the user is bored, sad, depressed, or tired. Low arousal and positive valence means that the user is calm, relaxed, or content. Stress indicates the user's level of emotional strain and pressure that the user is feeling in response to events or situations. Attention indicates the user's level of mentally concentrating on particular information while ignoring other information. This level of focalization of consciousness also indicates how easily the user's mind might be distracted by other stimuli, tasks, or information. Other cognitive states and physiological states can also be predicted by the neuroergonomic service 210.

Accordingly, the neuroergonomic service 210 can use at least some of the sensor data 208 of users (e.g., HRV, heart rate, EEG readings, body temperature, respiration rate, and/or pupil size, etc.) to infer their cognitive load, affect, stress, and/or attention. The availability of the type and the number of the sensor data 208 is a factor in the availability and the accuracy of the cognitive states that can be predicted by the neuroergonomic service 210. That is, if a user activates and makes available more of the sensors 206 for inferring her cognitive states, then the neuroergonomic service 210 can output a more comprehensive and holistic view of her physiological condition and psychological state.

The biosensing data 212 output by the neuroergonomic service 210 can include the sensor data 208 and/or the cognitive states. The biosensing data 212 can include metadata, such as timestamps, user identifiers, location data, device identifiers, session identifiers, etc., including any of the metadata included in the sensor data 208. The timestamps can be used to form a timeline of the biosensing data 212, for example, a trend graph line of a cognitive state over time.

In one implementation, the neuroergonomic service 210 calculates and outputs group metrics. For example, the neuroergonomic service 210 can aggregate the heart data for multiple users, and calculate and provide an average heart rate, a minimum heart rate, a maximum heart rate, a median heart rate, a mode heart rate, etc., of all users involved in a session.

In some implementations, the neuroergonomic service 210 can provide the biosensing data 212 in real time. That is, there is very little delay (e.g., seconds or even less than one second) from the time the sensors 206 take measurements to the time the neuroergonomic service 210 outputs the biosensing data 212. Therefore, the biosensing data 212 includes the current cognitive states of users based on real-time sensor readings. Additionally or alternatively, the neuroergonomic service 210 can output the biosensing data 212 that represents historical sensor readings and historical cognitive states.

The backend 202 of the neuroergonomic system 200 includes a contextual service 214. The contextual service 214 outputs contextual data 216. If users opt in, then the contextual service 214 tracks events that are affecting the users. For example, if a user is listening to a speech, then the contextual service 214 can convert the audio of the speech into text and/or sound envelope. The contextual data 216 can include a transcription of the speech along with timestamps. The contextual data can also include marks for noteworthy events (e.g., event markers, bookmarks, or flags), such as when the speech started and ended, when the speaker took breaks and resumed, when the crowded cheered, when certain keywords were spoken, etc. If a user is playing a video game, then the contextual service 214 can track events in the video game, such as progressions in gameplay and inputs from the user. The contextual data 216 can include video clips or screenshots of the video game, event markers (e.g., bonus points earned, leveling up, defeating a boss, etc.), indications of user inputs, timestamps, indications of players joining or leaving, etc. If a user is participating in a videoconference meeting, then the contextual service 214 can track events during the virtual meeting, such as words spoken by the participants, files or presentations shared during the meeting, participants joining and leaving the meeting, etc. If a user is shopping online, then the contextual service 214 can track events (including GUI events) during the shopping session, such as user inputs that browse through product selections, product categories and color choice options viewed; advertisements that popped up and clicked on or closed; items added to the cart; etc. These are but a few examples. Many different types of context are possible.

The contextual data 216 can include videos (e.g., video clips of a videoconference meeting or video clips of a movie), images (e.g., screenshots of a videoconference meeting or screenshots of video gameplay), audio (e.g., recordings of a speech or recordings of a videoconference meeting), texts (e.g., a transcription of a speech or a transcript of a conversation), files, and event markers. In some implementations, the contextual data 216 includes metadata, such as timestamps, identities of users, location data, device identifiers, session identifiers, context descriptions, etc. The timestamps can be used to form a timeline of events.

In some implementations, the contextual service 214 receives events and event-related information. For example, with video game players' permission, a video game server can be configured to automatically send game-related information to the contextual service 214 via APIs. The game-related information can include events along with timestamps, user inputs, game statistics, user identifiers, screenshots, etc. With meeting participants' permission, a videoconferencing server or a videoconferencing application can automatically send meeting-related information to the contextual service 214. The meeting-related information can include audio of conversations, a text transcription of conversations, chat history, timestamps, a list of participants, video recordings of the participants, screenshots of the meeting, etc.

Alternatively or additionally, a user can be enabled to manually add bookmarks to highlight noteworthy events either live as the events are happening or at a later time after the events have occurred. In some implementations, a user can configure or program the contextual service 214 to capture and/or bookmark specific events automatically. For example, a user can request that the contextual service 214 bookmark every time anyone in a videoconference meeting speaks a specific keyword (e.g., the user's name, a project name, or a client's name) or every time anyone joins or leaves. As another example, a user can request that the contextual service 214 bookmark every time any player levels up in a multiplayer video game. A variety of triggers for a bookmark are possible. This event information can be sent to the contextual service 214 in real-time or as a historical log of events.

In some implementations, the contextual data 216 (as well as the sensor data 208 and the biosensing data 212) can be logically divided into sessions. For example, a 30-minute videoconference meeting can constitute a session, 10 minutes of composing an email can constitute a session, an hour-long playing of a video game can constitute a session, watching a 1.5 hour-long movie can constitute a session, a 55-minute university class lecture can constitute a session, and so on.

Sessions can be automatically started and stopped. For example, a new session can start when a multiplayer video game begins, and the session can terminate when the video game ends. A new session can begin when the first participant joins or starts a videoconference meeting, and the session can end when the last participant leaves or ends the meeting. Session starting points and sessions end points can be manually set by a user. For example, a user can provide inputs to create a new session, end a session, or pause and resume a session. A live session may have a maximum limit on the size of the session window, depending on how much data can or is desired to be saved. A live session can have a rolling window where new data is added while old data is expunged or archived.

The frontend 204 of the neuroergonomic system 200 includes a neuroergonomic application 218. Although the neuroergonomic application 218 will be described as an application, it could be a service instead. The neuroergonomic application 218 receives the biosensing data 212 (e.g., sensor data and cognitive state data) and the contextual data 216 (e.g., event data) from the backend 202, and presents the data to users. The neuroergonomic application 218 displays the data in an intuitive way such that users can easily understand the correlation between the biosensing data 212 and the contextual data 216, and draw more useful insights than being presented with biosensing data 212 alone without the contextual data 216.

In some implementations, the neuroergonomic application 218 includes a unification module 220. The unification module 220 can receive the biosensing data 212 and the contextual data 216 from the backend services, for example, using API services. That is, the neuroergonomic service 210 and/or the contextual service 214 can push data to the neuroergonomic application 218, for example, in a live real-time streaming fashion or in a historical reporting fashion. Alternatively, the neuroergonomic application 218 can pull the data from the backend services, for example, periodically, upon a triggering event, or upon request by a user. The neuroergonomic service 210 and the contextual service 214 need not be aware that their data will later be aggregated by the neuroergonomic application 218. The availability and/or the breadth of the biosensing data 212 depends on the sensors 206 that the user has chosen to activate and/or the states that the machine learning models of the neuroergonomic service 210 are able to predict.

The unification module 220 aggregates the biosensing data 212 and the contextual data 216, for example, using metadata such as timestamps and user identifiers, etc. The aggregating can involve combining, associating, synchronizing, or correlating individual pieces of data in the biosensing data 212 with individual pieces of data in the contextual data 216. For instance, the unification module 220 can determine a correlation between the biosensing data 212 and the contextual data 216 based on the timestamps in the biosensing data 212 and the timestamps in the contextual data 216. The unification module 220 can also determine a correlation between the biosensing data 212 and the contextual data 216 based on user identifiers in the biosensing data 212 and user identifiers in the contextual data 216. Therefore, the unification module 220 is able to line up specific measurements associated with specific users at specific times in the biosensing data 212 (e.g., heart rate or stress level, etc.) with specific events associated with specific users at specific times in the contextual data 216 (e.g., leveling up in a video game or a protagonist character defeating an antagonist character in movie, etc.).

Alternative to the above-described implementation where the neuroergonomic service 210 calculates group metrics, in an alternative implementation, the unification module 220 of the neuroergonomic application 218 can calculate group metrics by aggregating the individual metrics. That is, the unification module 220 receives the biosensing data 212 associated with individual users and then computes, for example, average, mode, median, minimum, and/or maximum group statistics.

In some implementations, the neuroergonomic application 218 includes a presentation module 222. The presentation module 222 generates presentations and displays the presentations that include the biosensing data 212 in conjunction with the contextual data 216. The presentation module 222 can generate GUI components that graphically present certain biosensing measurements in the biosensing data 212 along with relevant events in the contextual data 216, such that a user can gain useful insights from the presentation.

For example, if a user permits her heart rate to be measured, then the presentation module 222 can generate a GUI component that displays the user's current heart rate that updates at certain intervals. If other users decide to share their heart rates with the user, then the presentation module 222 can generate one or more GUI components that display the other users' heart rates as well. The presentation module 222 can also display an aggregate (e.g., average/mean, mode, median, minimum, maximum, etc.) of the heart rates of the group of users. The presentation module 222 can display the current heart rates in real time or display past heart rates in a historical report. Similar presentations can be generated for other biosensing measurements, such as body temperature, EEG spectral band power, respiration rate, cognitive load, stress level, affective state, and attention level.

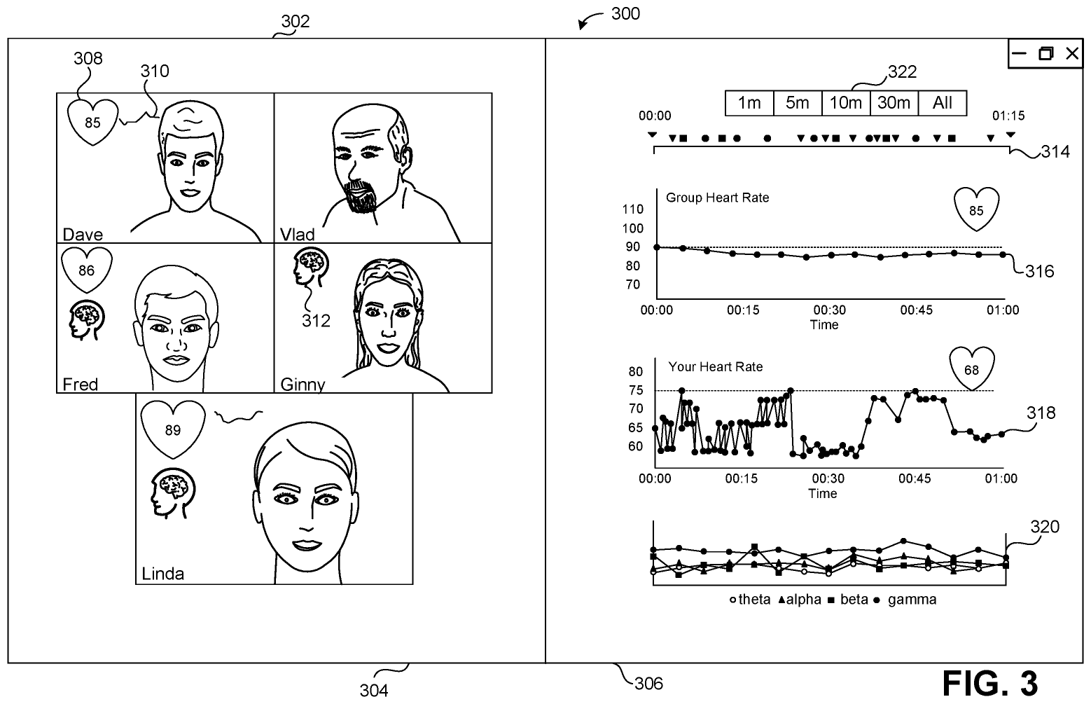

Furthermore, consistent with the present concepts, the presentation module 222 can generate displays that present biosensing measurements in context along with relevant events (e.g., using a timeline) so that the user can easily correlate the biosensing measurements with specific triggers. Examples of presentations (including display primitives) that the presentation module 222 can generate and use will be explained below in connection with FIGS. 3-9.

In some implementations, the presentation module 222 can provide alerts and/or notifications to the user. For example, if a biosensing measurement surpasses a threshold (e.g., the user's heart rate is above or below a threshold, or the user's cognitive load is above a threshold), the presentation module 222 highlights the biosensing measurement. Highlighting can involve enlarging the size of the GUI component that is displaying the biosensing measurement; moving the GUI component towards the center of the display; coloring, flashing, bordering, shading, or brightening the GUI component; popping up a notification dialog box; playing an audio alert; or any other means of drawing the user's attention.

In one implementation, the presentation module 222 can highlight the GUI component that is displaying the event that corresponds to (e.g., has a causal relationship with) the biosensing measurement that surpassed the threshold. For example, if a participant in a videoconference meeting shares new content that causes the user's heart rate to rise in excitement, then the presentation module 222 can highlight both the user's high heart rate on the display and the video feed of the shared content on the display. Highlighting both the biosensing data 212 and the contextual data 216 that correspond to each other will enable the user to more easily determine which specific event caused which specific biosensing measurement.

In some implementations, the format of the presentations generated by the presentation module 222 is dependent on the availability and the types of biosensing data 212 being displayed; the availability and the types of contextual data 216 being displayed; the user's preferences on the types of data and the types of GUI components she prefers to see; and/or the available display size to fit all the data. In some implementations, the presentations generated by the presentation module 222 are interactive. That is, the user can provide inputs to effect changes to the presentations. For example, the user can select which biosensing measurements to display. The user can choose the ordering of the biosensing measurements as well as choose which biosensing measurements to display more or less prominently. The user can provide inputs to select for which of the other users the presentation should show biosensing measurements, assuming those other users gave permission and shared their data. Furthermore, the user can provide input to choose a time window within which the biosensing data 212 and the contextual data 216 will be displayed. For example, if a video game session is one-hour long, the user can choose a particular 5-minute time segment for which data will be displayed.

In some implementations, the neuroergonomic application 218 includes a recommendation module 224. The recommendation module 224 formulates a recommendation based on the biosensing data 212 and/or the contextual data 216. The recommendation module 224, in conjunction with the presentation module 222, can present a recommendation to a user and/or execute the recommendation.

A recommendation can include an intervening action that brings about some positive effect and/or prevents or reduces negative outcomes. For example, if one or more participants in a videoconference meeting are experiencing high levels of stress, then the recommendation module 224 can suggest that the participants take deep breaths, take a short break, or reschedule the meeting to another time, rather than continuing the meeting that is harmful to their wellbeing. If students in a classroom are experiencing boredom, then the recommendation module 224 can suggest to the teacher to change the subject, take a recess, add exciting audio or video presentations to the lecture, etc. The '022 application, identified above as being incorporated by reference herein, explains example techniques for formulating, presenting, and executing the recommendations.

The recommendations can be presented to users visually, auditorily, haptically, or via any other means. For example, a recommendation to take a break can be presented via a popup window or a dialog box on a GUI, spoken out loud via a speaker, indicated by warning sound, indicated by vibrations (e.g., a vibrating smartphone or a vibrating steering wheel), etc.

The recommendation module 224 can receive an input from the user. The input from the user may indicate an approval or a disapproval of the recommended course of action. Even the absence of user input, such as when the user ignores the recommendation, can indicate a disapproval. The recommendation module 224 can execute the recommended course of action in response to the user's input that approves the action.

### Real-Time Report

FIG. 3 illustrates an example live presentation 300, consistent with some implementations of the present concepts. In this example, the live presentation 300 includes a videoconferencing application 302 that enables a user (e.g., Linda) to engage in a virtual meeting with other participants (e.g., Dave, Vlad, Fred, and Ginny). The videoconferencing application 302 includes a participants pane 304 on the left side that shows a live video feed from each participant, and also includes a statistics pane 306 on the right side. Both of the participants pane 304 and the statistics pane 306 can present biosensing data in conjunction with contextual data.

Conventional videoconferencing applications typically enable the user, via menus, to choose whether to share or not share her video, audio, files, and/or screen with the other participants in the meeting. Consistent with the present concepts, the videoconferencing application 302 enables the user to choose to share (or not share) her biosensing measurements (e.g., heart rate, body temperature, cognitive load, stress, attention, etc.) with other participants in the meeting. The selection of the biosensing measurements that are available for the user to share with other participants depends on the availability of sensors, whether the user has opted in to have the sensors take specific readings, and whether the user has permitted specific uses of the sensor readings.

In one implementation, the user can choose which specific biosensing measurements to share with which specific participants. That is, the user need not choose to share with all participants or none (i.e., all or nothing). The user can specify individuals with whom she is willing to share her biosensing measurements and specify individuals with whom she is unwilling to share her biosensing measurements. For example, an employee can share her biosensing measurements with her peer coworkers but not with her bosses. A video game player can share her biosensing measurements with her teammates but not with opposing team players.

In the example shown by the participants pane 304 in FIG. 3, Dave and Fred opted in to share their current heart rates 308 with Linda; Dave opted in to share his heart rate trend 310 with Linda; Fred and Ginny opted in to share their stress level 312 with Linda; and Vlad opted out of sharing his biosensing data with Linda. Accordingly, the videoconferencing application 302 overlays GUI components (e.g., symbols, icons, graphics, texts, etc.) that represent the available biosensing measurements of the participants on their video feeds.

The other participants may have shared their biosensing measurements with Linda specifically, with a larger group that includes Linda, or with everyone in the meeting. Vlad may have shared his biosensing measurements with other participants but not with Linda. In the example in FIG. 3, Linda can also view her own biosensing measurements. Some of the participants may not have the necessary sensors to detect certain physiological inputs and to infer certain cognitive states. Users with and without sensors can nonetheless participate in the virtual meeting.

The overlaid GUI components can be updated periodically (e.g., every 1 second, 10 seconds, 30 seconds, etc.) or can be updated as new measurements are received (via push or pull). Other GUI components that convey other biosensing measurements (e.g., cognitive load, affective state, attention level, mood, fatigue, respiration rate, EEG bands, body temperature, perspiration rate, etc.) are possible.

Accordingly, the participants pane 304 can present biosensing data (e.g., heart rates, heart rate trends, and stress levels) in conjunction with contextual data (e.g., videos of participants). Thus, the user (e.g., Linda) is able to visually correlate the biosensing measurements with specific events that occur concurrently. For example, if Fred starts assigning difficult projects with short deadlines, Linda may observe that the participants' heart rates and stress levels rise concurrently. As another example, if Vlad speaks slowly and quieting about a boring topic for an extended period of time, Linda may observe that the participants heart rates slow down.

The statistics pane 306 can also present contextual data and biosensing data associated with the user (e.g., Linda), other participants (e.g., Dave, Vlad, Fred, or Ginny), and/or the group. In the example shown in FIG. 3, the statistics pane 306 presents a timeline 314 of events drawn from the contextual data as well as a group heart rate 316, the user's heart rate 318, and the user's EEG band powers 320 drawn from the biosensing data.

The statistics pane 306 can include controls 322 for adjusting the time axes of one or more of the GUI components. In one implementation, the controls 322 can change the time axis scale for one or more of the GUI components. In another implementation, the controls 322 allow the user to increase or decrease the time range for the contextual data and/or the biosensing data displayed in the statistics pane 306. The time axes of the multiple GUI components can be changed together or individually. Presenting the contextual data and the biosensing data on a common timeline (e.g., the x-axes having the same range and scale) can help the user more easily determine the causal relationships between the specific events in the contextual data and the specific measurements in the biosensing data.

In addition to the metrics associated with Linda, the statistics pane 306 can also display individual metrics associated with any other participant (e.g., Dave, Vlad, Fred, or Ginny). The statistics pane 306 can also display group metrics, such as mean, median, mode, minimum, maximum, etc. Each participant can choose not to share her individual metrics from other participants for privacy purposes but still share her individual metrics for the calculation of group metrics. This level of sharing may be possible only where there are enough individuals sharing their metrics so that the group metrics do not reveal the identities of any individual (e.g., where there are only two participants). Accordingly, participants may be able to gain insights as to how the group is reacting to certain events during the virtual meeting without knowing how any specific individual reacted to the events.

The combination of biosensing data and contextual data displayed in the participants pane 304, the statistics pane 306, or both, allows the observer to visually correlate specific biosensing measurements with specific events that occurred concurrently. Thus, insights regarding the causes or triggers for the changes in biosensing measurements can be easily determined. For example, if Linda notices a particular participant or multiple participants experience high stress, elevated cognitive load, raised heart rate, etc., then Linda should immediately be able to determine what specific event (e.g., the CEO joining the meeting, a difficult project being assigned to an inexperienced employee, the meeting running over the allotted time, etc.) caused such responses in the participants.

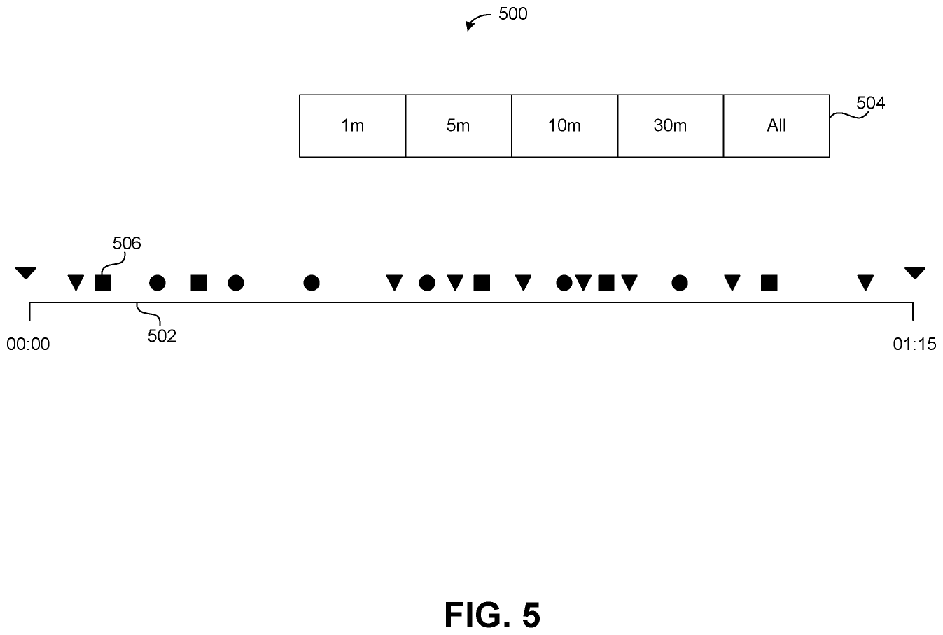

The data presented in the statistics pane 306 can be updated periodically (e.g., every 1 second, 10 seconds, 30 seconds, etc.) or can be updated as new measurements are received (via push or pull). Other GUI components that convey other contextual data (e.g., screenshots or transcripts) and other biosensing measurements (e.g., cognitive load, affective state, attention level, mood, fatigue, respiration rate, body temperature, perspiration rate, etc.) are possible. These and other examples of display primitives for presenting contextual data and biosensing data will be described below in connection with FIGS. 5-9.

In some implementations, the videoconferencing application 302 can display recommendations in real-time (e.g., during the virtual meeting). The recommendations can include a dialog box that suggests, for example, taking deep breaths, turning on meditative sounds, taking a break from the meeting, stretching, etc. The videoconferencing application 302 can highlight the biosensing measurements that triggered the recommendation, for example, high group stress level or rising heart rates.

Although the live presentation 300 has been described above in connection with the example videoconferencing application 302, the live presentation 300 can be incorporated into other applications, such as video games, word processors, movie players, online shopping websites, virtual classrooms, vehicle navigation consoles, virtual reality headgear or glasses, etc. Any existing applications can be modified to function as a neuroergonomic application that receives biosensing data and contextual data, unifies the data, generates presentations of the data, and/or displays the data to users.

Accordingly, the live presentation 300 gives the user real-time insights about the user herself and the other users as events are happening. That is, the user can view sensor measurements and cognitive states of the group of participants, and correlate the changes in such biosensing metrics with live events. Thus, the user can gain immediate insights into how the group is reacting to specific events as they occur. For example, a lecturer can gain real-time insights into how her students are reacting to the subject of the lecture; a speaker can gain real-time insights into how the audience is responding to the words spoken, an advertiser can gain real-time insights into how the target audience is responding to specific advertisements, a writer can track her real-time cognitive states as she is writing, etc.

Such real-time feedback can enable a user to intervene and take certain actions to improve the participants' wellbeing, reduce negative effects, and/or promote and improve certain products or services. For example, a disc jockey can change the music selection if the listeners are getting bored of the current song, an employee can initiate a break if she is experiencing high cognitive load, a movie viewer can turn off the horror movie if she is experiencing high heart rate, etc. Many other intervening actions and benefits are possible.

### Summary Report

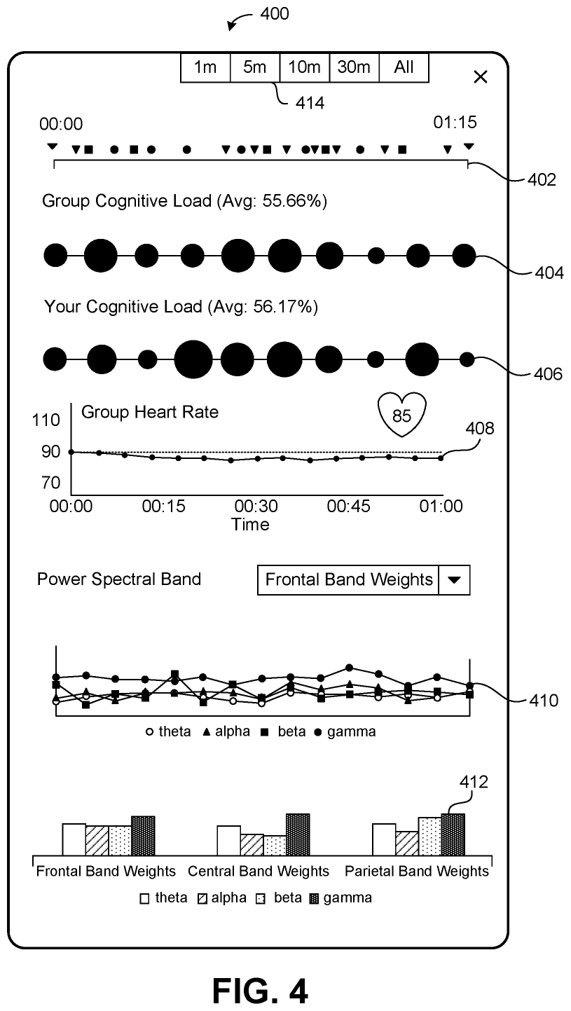

FIG. 4 illustrates an example historical presentation 400, consistent with some implementations of the present concepts. The historical presentation 400 includes a summary report that presents contextual data and/or biosensing data associated with one or more users. In the example shown in FIG. 4, the historical presentation 400 includes a timeline 402 of events in the contextual data as well as a group cognitive load 404, a user's cognitive load 406, a group heart rate 408, an EEG power spectral band graph 410 of the frontal band weights, and a power spectral band graph 412 of band weights for multiple brain regions in the biosensing data.

In some implementations, the contextual data and the biosensing data that were received and combined to generate the live presentation 300, described above in connection with FIG. 3, are the same data stored and presented in the historical presentation 400, except that the historical presentation 400 shows past history of measurements, whereas the live presentation 300 shows current real-time measurements. Nonetheless, the sources of the data and GUI components used to present the data can be the same for both the live presentation 300 and the historical presentation 400.

The historical presentation 400 can include controls 414 for adjusting the scales or the ranges of the time axes for one or more of the GUI components in the historical presentation 400. For example, selecting “1 minute,” “5 minutes,” “10 minutes,” “30 minutes,” or “All” option can change the GUI components in the historical presentation 400 to show the contextual data and a trend of the biosensing data within only the selected time window. Alternatively or additionally, the user may be enabled to select a time increment rather than a time window. Furthermore, the timeline 402 can include an adjustable slider that the user can slide between the displayed time window to view the contextual data and the biosensing data within a desired time segment. Many options are possible for enabling the user to navigate and view the desired data.

The frequency of the contextual data points and/or the biosensing data points depends on the availability of data received (either pushed or pulled). For example, if an individual heart rate was measured periodically at a specific frequency (e.g., every 1 second, 10 seconds, 30 seconds, etc.), then the heart rate data included in the historical presentation 400 would include the sampled heart rate data at the measured frequency.

Similar to the statistics pane 306 described above in connection with FIG. 3, the historical presentation 400 can display any group metrics (such as mean, median, mode, minimum, maximum, etc.) for any of the biosensing data. Other combinations of specific contextual data and/or specific biosensing data can be presented in the historical presentation 400. For example, screenshots and/or transcripts from the contextual data can be presented along with the timeline 402. Other biosensing measurements (e.g., cognitive load, affective state, attention level, mood, fatigue, respiration rate, body temperature, perspiration rate, etc.) can be included in the historical presentation 400. The example display primitives in the historical presentation 400 as well as other example display primitives will be described below in connection with FIGS. 5-9.

Accordingly, the historical presentation 400 gives the user a history of insights about a group of participants in relation to specific events that occurred in synchronization with the biosensing data. That is, the user can visually analyze physiological measurements and cognitive states of the group of participants, and associate the changes in biosensing metrics with events that triggered those changes. Thus, the user can gain valuable insights into how the group reacted to specific events by analyzing the historical data, and use those insights to improve products or services that will generate better stimuli in the future.

For example, a video game developer can measure neuroergonomic responses of players during various stages of a video game and modify aspects of the video game to eliminate parts that caused boredom, anger, or stress, while enhancing parts that elicited happiness, content, arousal, excitement, or attention. A web designer can measure neuroergonomic responses of website visitors and improve the website by removing aspects of the website that caused negative affective states. Advertisers, film editors, toy designers, book writers, and many others can analyze the historical presentation 400 of willing and consensual test subjects to improve and enhance advertisements, films, toys, books, and any other products or services. Workplace managers can use the historical presentation 400 to determine which projects or tasks performed by employees caused negative or positive responses among the employees. A classroom teacher can analyze how her students responded to different subjects taught and various tasks her students performed throughout the day. A yoga instructor can split test (i.e., A/B test) multiple meditative routines to determine which routine is more calming, soothing, and relaxing for her students. A speech writer can analyze whether the audience had positive or negative responses to certain topics or statements, and revise her speech accordingly. The present concepts have a wide array of applications in many fields.

The historical presentation 400 can provide detailed contextual information (e.g., which specific event) that triggered certain neuroergonomic responses. By synchronizing the biosensing data with the contextual data in the historical presentation 400, the user can determine which physiological changes were induced by which external stimuli. For example, the user can determine which part of a speech or a meeting triggered a certain emotional response among the audience or the meeting participants, respectively. Furthermore, the user can determine which scene in a movie caused the audience's heart rate to jump. Additionally, the user can determine the cognitive load level associated with various parts of a scholastic test. The neuroergonomic insights along with background context provided by the present concepts can be used to improve user wellbeing as well as to improve products and services.

### Display Primitives

The present concepts include visualizations for presenting biosensing data and contextual data. Application developers can design and/or use any GUI components to display biosensing data and contextual data to users. Below are some examples of display primitives that can be employed to effectively communicate neuroergonomic insights along with context to users. Variations of these examples and other display primitives can be used. The below display primitives can be integrated into any application GUI.

Furthermore, a software development kit (SDK) may be available for software developers to build, modify, and configure applications to use the outputs from the neuroergonomic service and/or the contextual service, generate presentations, and display the presentations to users. The SDK can include the display primitives described below as templates that software developers can use to create presentations and GUIs.

FIG. 5 illustrates an example context display primitive 500, consistent with some implementations of the present concepts. The context display primitive 500 can be used to present contextual data. The context display primitive 500 includes a timeline 502 (e.g., an x-axis that represents time). The timeline 502 can span the entire period of time that encompasses the available contextual data (e.g., a session) or a portion thereof. The context display primitive 500 includes time controls 504 that can be selected by the user to change the period of time represented by the timeline 502. If the timeline 502 shows only a portion of the entire time period that represents the available contextual data, then the context display primitive 500 can display a slider or a bar that can be used to display different portions of the available time period.

The timeline 502 includes marks 506 (e.g., bookmarks or tick marks) that represent specific events. For example, the marks 506 can represent specific keywords spoken during a speech, a meeting, or a song; certain users joining or leaving a meeting; earning bonuses, leveling up, or dying in a video game; scene changes, cuts, or transitions in a movie; user inputs (e.g., keyboard inputs, mouse inputs, user interface actions, etc.) during a browsing session, a video game, or a virtual presentation; or specific advertisements presented during a web browsing session. Depending on the context and scenario, the marks 506 can indicate any event, product, service, action, etc.

Various GUI features can be incorporated into the context display primitive 500. In the example illustrated in FIG. 5, there are multiple classes of the marks 506, including circular marks, triangular marks, and square marks. These different classes can be used to indicate different types of events or different users associated with the events. Alternatively or additionally, the mark 506 can be displayed using different colors (e.g., red marks, yellow marks, green marks, etc.) to indicate various types of events. The marks 506 can be clickable, hoverable, or tappable to reveal more information about specific events. For example, the marks 506 may be activated to show details about the represented events, such as text descriptions of the events, screenshots, identities of people, timestamps, etc.

As discussed above in connection with FIG. 2, the events represented by the marks 506 can be captured by a backend contextual service. The events can be sent to the backend contextual service automatically by a program (e.g., an application or a service). For example, a video game server can automatically send certain significant events (e.g., loss of life, winning an award, advancing to the next level, high network latency, etc.) to the backend contextual service using API services. Alternatively or additionally, the events represented by the marks 506 can be manually set by a user. For example, a player can provide an input (e.g., a voice command or a button input on the game controller) to manually mark a noteworthy moment during gameplay.

The context display primitive 500 helps the user visualize the timeline of events graphically so that the simultaneous presentation of biosensing data can be better understood in context with the events that occurred concurrently. Consistent with the present concepts, presenting the context display primitive 500 along with biosensing data enables the user to better understand the biosensing data in the context informed by the context display primitive 500. For example, the user can visually align the biosensing data (including noteworthy changes in the biosensing data) with specific events or stimuli that caused the specific biosensing data. In some implementations, activating the time controls 504 to change the timeline 502 to display different portions of the available time period can also automatically change other display primitives that are presenting biosensing data to display matching time periods.

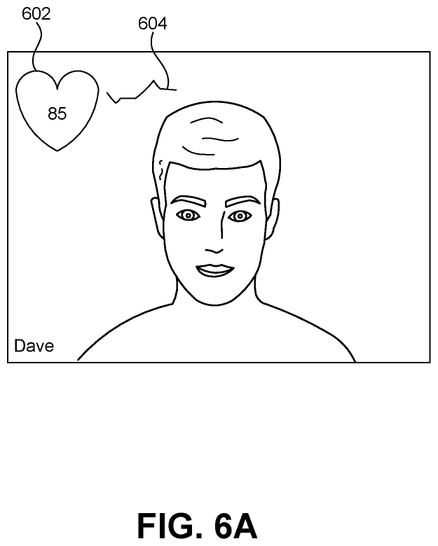

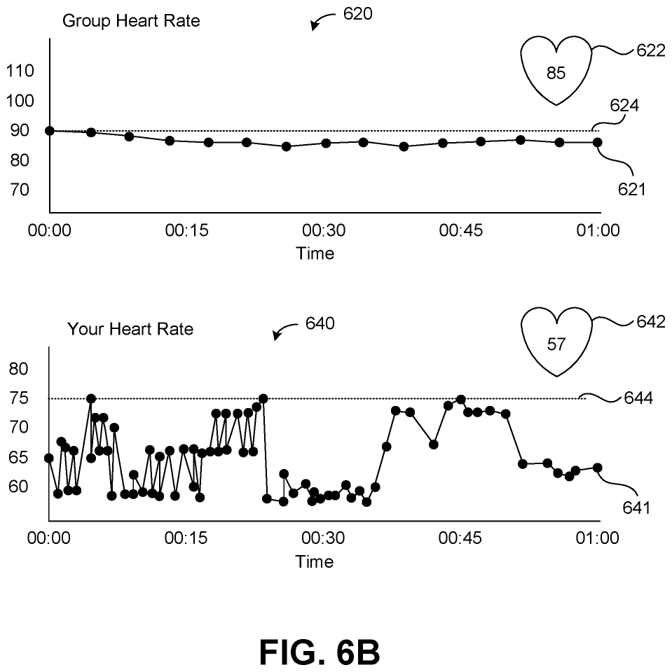

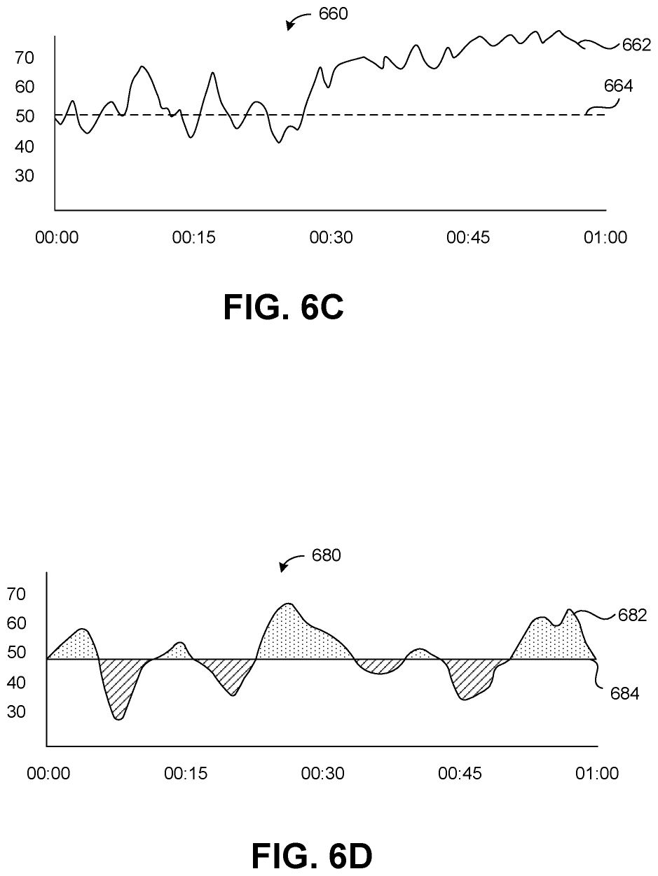

FIGS. 6A-6D illustrate example heart rate display primitives, consistent with some implementations of the present concepts. The heart rate display primitives can be used to present heart rate data in biosensing data.

FIG. 6A shows a heart symbol 602 with a numerical value representing the heart rate (e.g., in units of beats per minute). This heart rate can represent the current heart rate in a live presentation or a past heart rate at a specific point in time in a historical presentation. FIG. 6A also shows a heart rate trend line 604, which graphically presents the heart rate measurements taken over a period of time. Although the axes are not drawn, the x-axis represents time and the y-axis represents the heart rate.

The heart symbol 602 and/or the heart rate trend line 604 can be displayed to a user in isolation or can be overlaid (as shown in FIG. 6A). For example, the heart symbol 602 and/or the heart rate trend line 604 can be overlaid on top of the video feed of a participant in a videoconference meeting, near an avatar of a video game player, next to a list of users, etc. The heart rate displayed inside the heart symbol 602 and the heart rate trend line 604 can represent the heart rate data of the user herself or of another user who has opted to share her heart rate with the user.

FIG. 6B includes a group heart rate graph 620 and a user heart rate graph 640. The group heart rate graph 620 shows a timeline of group heart rates. The group heart rate graph 620 includes a group heart rate trend line 621 of a group of users over a period of time. The group heart rate graph 620 includes a heart symbol 622 that includes a numerical value of the current heart rate or the latest heart rate in the displayed period of time. The group heart rate can be calculated by aggregating the individual heart rates of multiple users by any arithmetic method, such as mean, median, mode, minimum, maximum, etc. Furthermore, the group heart rate graph 620 includes a maximum line 624 to indicate the maximum group heart rate over the displayed period of time.

The user heart rate graph 640 shows a timeline of user heart rates. The user heart rate graph 640 includes a user heart rate trend line 641 of a user over a period of time. The user heart rate graph 640 includes a heart symbol 642 that includes a numerical value of the current heart rate or the latest heart rate in the displayed period of time. The user heart rate graph 640 includes a maximum line 644 to indicate the maximum user heart rate over the displayed period of time.

FIG. 6C includes a heart rate graph 660 that shows a heart rate trend line 662 in comparison to a baseline heart rate line 664. The heart rate graph 660 in FIG. 6C allows the user to visually determine whether the current heart rate or the heart rate at a particular point in time is at, above, or below the user's baseline heart rate. FIG. 6D includes a heart rate graph 680 that further highlights whether a heart rate trend line 682 is above or below a baseline heart rate line 684 using different shades or colors.

Consistent with the present concepts, presenting a heart rate display primitive along with contextual data enables the user to better understand the heart rate data in the context informed by the contextual data. For example, the user can visually align the heart rate data (including noteworthy changes in a person's heart rate) with specific events or stimuli that caused the heart rate to rise or fall.

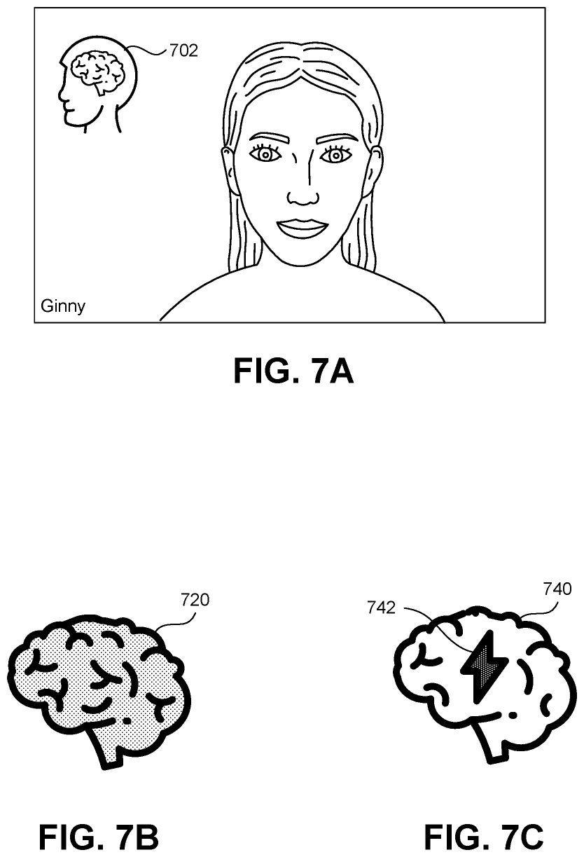

FIGS. 7A-7C illustrate example cognitive state display primitives, consistent with some implementations of the present concepts. The cognitive state display primitives can be used to present cognitive state data in biosensing data, such as cognitive load level, stress level, affective state, and attention level.

FIG. 7A shows a brain symbol 702 representing a cognitive state of the user (Ginny in this example). The brain symbol 702 can vary in color, vary in size, have different text inside, have different shading, include various icons, etc., to indicate any one or more of cognitive load levels, stress levels, affective states, and attention levels. The brain symbol 702 can be displayed to a user in isolation or can be overlaid (as shown in FIG. 7A) on top of another GUI component. For example, the brain symbol 702 can be overlaid on top of the video feed of a participant in a videoconference meeting or displayed near an avatar of a video game player, etc.

FIG. 7B shows a brain symbol 720 whose shading or coloring can indicate various cognitive states of a user. For example, a green color can indicate a low stress level, a yellow color can indicate a medium stress level, and a red color can indicate a high stress level. In one implementation, the brain symbol 720 can be divided into two parts or into four parts to indicate additional cognitive states. FIG. 7C shows a brain symbol 740 with an icon 742 inside. The icon 742 can indicate a particular cognitive state. Although FIG. 7C shows the icon 742 as a lightning symbol, other graphical components are possible, such as circles, triangles, squares, stars, emoji faces, numbers, text, etc. The icon 742 can vary in shape, size, color, shading, highlighting, blinking, flashing, etc., to indicate various cognitive states. Furthermore, the brain symbol 720 in FIG. 7B and the brain symbol 740 in FIG. 7C can be combined, such that the brain symbol 720 can be shaded in multiple colors and also include the icon 742 with its own variations. The numerous permutations of presenting the combination of the brain symbol 720 and the icon 742 are sufficiently high enough to visually convey multiple cognitive states that are possible.

Consistent with the present concepts, presenting a cognitive state display primitive along with contextual data enables the user to better understand the cognitive state data in the context informed by the contextual data. For example, the user can visually correlate the cognitive state data (including noteworthy changes in a person's cognitive state) with specific events or stimuli that caused the specific cognitive state.

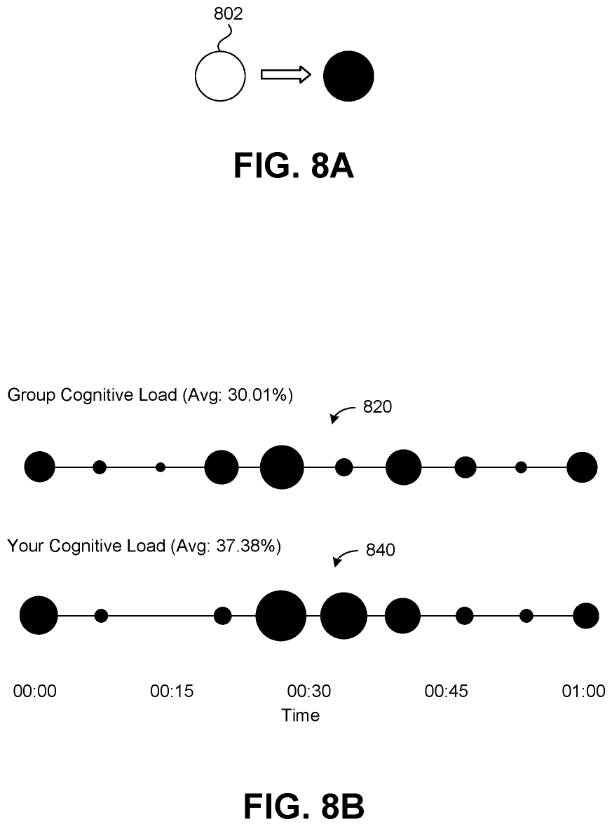

FIGS. 8A and 8B illustrate example cognitive load display primitives, consistent with some implementations of the present concepts. The cognitive load display primitives can be used to present cognitive load data in biosensing data. For example, where the cognitive load ranges from an engineered score of 0% to 100%, a cognitive load display primitive can present the cognitive load value in a numerical format or in a graphical format, such as a bar graph.

FIG. 8A shows a cognitive load indicator 802. The cognitive load indicator 802 can vary in color, vary in size, vary in shape, have different text inside, have different shading, include various icons, etc., to indicate the cognitive load metrics associated with a user. For example, in FIG. 8A, a white color indicates low cognitive load, whereas a black color indicates a high cognitive load. Many other variations are possible. For example, colors green, yellow, and red can be used to indicate low, medium, and high cognitive loads, respectively. Or, a gray shade gradient can be used to indicate more granular variations in the cognitive load levels. The cognitive load indicator 802 can be displayed to a user in isolation or can be overlaid on top of a video feed of a participant in a videoconference meeting or displayed near an avatar of a video game player, etc.

FIG. 8B includes a group cognitive load graph 820 and a user cognitive load graph 840. The group cognitive load graph 820 shows a timeline of the cognitive load level trend of a group of users over a period of time. The cognitive load levels are indicated by the sizes of the circles, where smaller circles reflect lower cognitive loads, and larger circles reflect higher cognitive loads. The group cognitive load graph 820 displays the average cognitive load for the group using text (i.e., 30.01% in the example shown in FIG. 8B). This average cognitive load value can be the average over the time period current displayed by the group cognitive load graph 820 or over the time period spanning the entire session. The group cognitive load level can be an aggregate of the individual cognitive load levels of multiple users using any arithmetic method, such as mean, median, mode, minimum, maximum, etc.

The user cognitive load graph 840 shows a timeline of the cognitive load level trend of a user over a period of time. The user cognitive load graph 840 displays the average cognitive load for the user using text (i.e., 37.38% in the example shown in FIG. 8B).

Other variations in the presentation of the cognitive load data are possible. For example, any cognitive load measurement that is above a certain threshold (e.g., 70%) may be highlighted by a red colored circle or by a flashing circle as a warning that the cognitive load level is high. Each of the circles may be selectable to reveal more details regarding the cognitive load measurement. The frequency of cognitive load measurements can vary. The circles in the graphs can move left as new cognitive load measurements are presented on the far right-hand side of the graphs.

Consistent with the present concepts, presenting a cognitive load display primitive along with contextual data enables the user to better understand the cognitive load data in the context informed by the contextual data. For example, the user can visually match the cognitive load levels (including noteworthy changes in a person's cognitive load level) with specific events or stimuli that caused the specific cognitive load level.

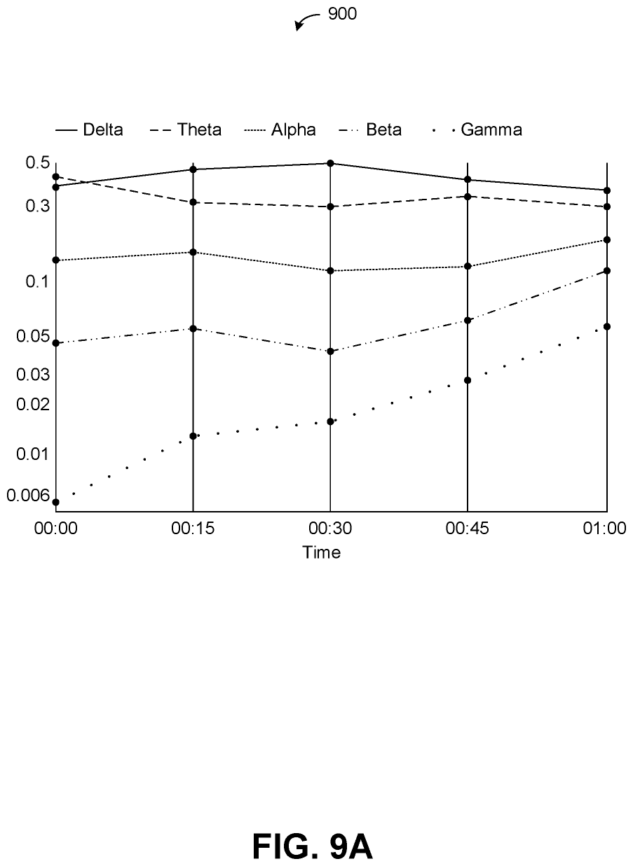

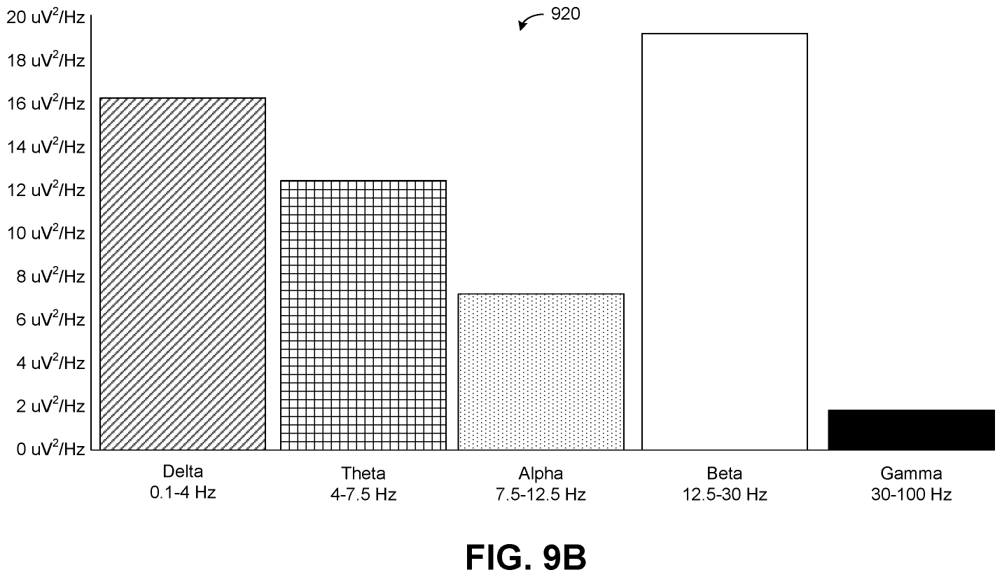

FIGS. 9A and 9B illustrate example EEG display primitives, consistent with some implementations of the present concepts. The EEG display primitives can be used to present EEG data in biosensing data.

FIG. 9A includes an EEG trend graph 900 that shows a timeline of the EEG power spectral band readings of a user over a period of time for the delta, theta, alpha, beta, and gamma bands. The EEG trend graph 900 can vary in many ways, including the scales and units of the axes, the frequency in which measurements are taken, thickness and/or color of the trend lines, etc. The EEG trend graph 900 visually shows how the EEG power spectral bands change over time. FIG. 9B includes an EEG band graph 920 that shows the relative power of the multiple bands (i.e., delta, theta, alpha, beta, and gamma bands) at a point in time or over a window of time. Similar to the EEG trend graph 900, the y-axis in the EEG band graph 920 represents power. The EEG band graph 920 can vary in many ways. Similar to the EEG power spectral band graph 410 show in FIG. 4, the EEG trend graph 900 and/or the EEG band graph 920 in FIG. 9 can include a selector (e.g., a drop down list menu or a radio button menu) to display the EEG band readings from different regions of the brain (e.g., frontal, parietal, left, right, etc.).

Consistent with the present concepts, presenting an EEG display primitive along with contextual data enables the user to better understand the EEG data in the context informed by the contextual data. For example, the user can visually associate the EEG power levels with specific events or stimuli that caused the specific EEG power levels.

### Processes

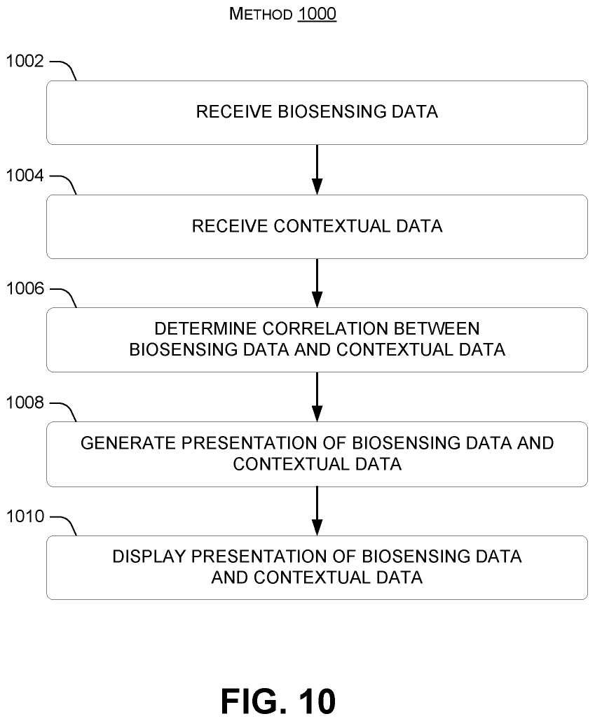

FIG. 10 illustrates a flowchart of an example neuroergonomic method 1000, consistent with some implementations of the present concepts. The neuroergonomic method 1000 is presented for illustration purposes and is not meant to be exhaustive or limiting. The acts in the neuroergonomic method 1000 may be performed in the order presented, in a different order, or in parallel or simultaneously, may be omitted, and may include intermediary acts therebetween.

In act 1002, biosensing data is received. The biosensing data can be pushed or pulled, for example, via an API service. In some implementations, the biosensing data is provided by a neuroergonomic service that outputs, for example, sensor data measured by sensors and/or cognitive state data inferred by machine learning models. The sensor data can include, for example, heart rates, EEG spectral band powers, body temperatures, respiration rates, perspiration rates, pupil size, skin tone, motion data, ambient lighting, ambient sounds, video data, image data, audio data, etc., associated with one or more users. The cognitive state data can include, for example, cognitive load level, stress level, attention level, affective state, etc., associated with one or more users. The types of biosensing data that are received depend on the set of sensors available and activated as well as the individual user's privacy setting indicating which data types and which data uses have been authorized.

In some implementations, the biosensing data includes metadata, such as time data (e.g., timestamps) and/or user identifiers associated with the biosensing data. That is, each sensor measurement and each cognitive state prediction can be associated with a specific user and a timestamp. For example, the biosensing data can indicate that Linda's hear rate is 85 beats per minute at 2022/01/31, 09:14:53 PM or Dave's cognitive load level is 35% at 2020/12/25, 11:49:07 AM.

In act 1004, contextual data is received. The contextual data can be pushed or pulled, for example, via an API service. The contextual data can be provided by a server or an application. For example, a game server or a game application can provide game-related events during a session of a video game. A web server or a web browser application can provide browsing events during an Internet browsing session. A videoconferencing server or a videoconferencing application can provide events related to a virtual meeting. A video streaming server or a movie player application can provide events during a movie-watching session. The contextual data can include video, image, audio, and/or text.

In some implementations, the contextual data includes metadata, such as time data (e.g., timestamps) and/or user identifiers associated with the contextual data. That is, each event can be associated with a specific user and a timestamp. For example, an example event can indicate that Linda joined a meeting, Dave stopped playing a video game, Ginny added a product to her online shopping cart, Fred closed a popup advertisement, etc.

In act 1006, correlations between the biosensing data and the contextual data are determined. In some implementations, the biosensing data and the contextual data are aligned with each other based on the timestamps in the biosensing data and the timestamps in the contextual data. Additionally, in some implementations, the biosensing data and the contextual data are associated with each other based on the user identifiers in the biosensing data and the user identifiers in the contextual data.

Accordingly, the biosensing data is placed in a common timeline with the contextual data, such that the biosensing data can make more sense in the context of concurrent events that coincide with the sensor data and/or the cognitive state data. Therefore, consistent with the present concepts, the combination of the biosensing data and the contextual data provides greater insights than viewing the biosensing data without the contextual data.

In act 1008, a presentation of the biosensing data and the contextual data is generated. For example, a GUI presentation that displays both the biosensing data and the contextual data can be generated by an application (e.g., a browser client, a videoconferencing app, a movie player, a podcast app, a video game application, etc.). In some implementations, the presentation can use the example display primitives described above (e.g., the context display primitives, the heart rate display primitives, the cognitive state display primitives, the cognitive load display primitives, and the EEG display primitives) or any other graphical display elements.

In some implementations, the presentation can include audio elements and/or text elements. For example, the presentation can include an audible alert when a user's stress level is high or a textual recommendation for reducing the user's stress level.

In one implementation, the types of biosensing data and the types of contextual data that are included in the presentation as well as the arrangement and the format of the presented data can depend on user preferences, availability of data, and/or screen real estate. That is, any combination of the above examples of various types of biosensing data can be included in the presentation.

In act 1010, the presentation of the biosensing data and the contextual data is displayed. For example, a device and/or an application that the user is using can display the presentation to the user on a display screen. The audio portion of the presentation can be output to the user via a speaker. In some implementations, the presentation can be interactive. That is, the user can select and/or manipulate one or more elements of the presentation. For example, the user can change the time axis, the user can select which biosensing data to show, the user can obtain details about particular data, etc.

In one implementation, the neuroergonomic method 1000 is performed in real-time. For example, there is low latency (e.g., only seconds elapse) from taking measurements using sensors to presenting the biosensing data and the contextual data to the user. In another implementation, the presentation of the biosensing data and the contextual data occurs long after the sensor measurements and contextual events occurred.

### System Configurations

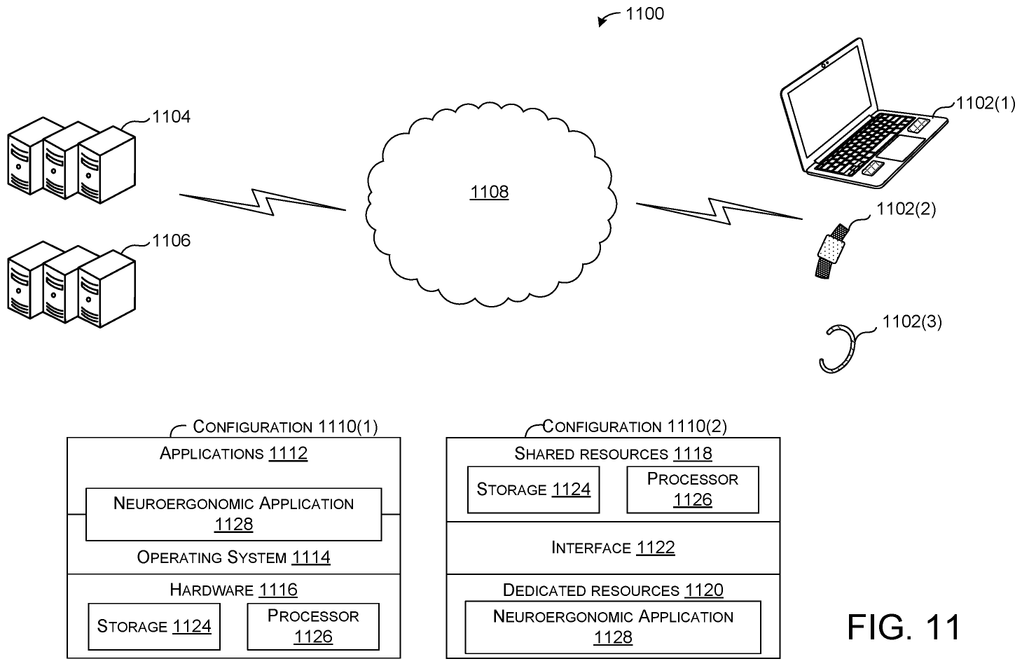

FIG. 11 illustrates example configurations of a neuroergonomic system 1100, consistent with some implementations of the present concepts. This example neuroergonomic system 1100 includes sensors 1102 for taking measurement inputs associated with a user. For example, a laptop 1102(1) includes a camera, a microphone, a keyboard, a touchpad, a touchscreen, an operating system, and applications for capturing physiological inputs, digital inputs, and/or environmental inputs associated with the user. A smartwatch 1102(2) includes biosensors for capturing the heart rate, respiration rate, perspiration rate, etc. An EEG sensor 1102(3) measures brain activity of the user. The sensors 1102 shown in FIG. 11 are mere examples. Many other types of sensors can be used to take various readings that relate to or affect the biosensing measurements that are desired.

The measured inputs are transferred to a neuroergonomic server 1104 through a network 1108. The network 1108 can include multiple networks and/or may include the Internet. The network 1108 can be wired and/or wireless.

In one implementation, the neuroergonomic server 1104 includes one or more server computers. The neuroergonomic server 1104 runs a neuroergonomic service that takes the inputs from the sensors 1102 and outputs biosensing data. For example, the neuroergonomic service uses machine learning models to predict the cognitive states of the user based on the multimodal inputs from the sensors 1102. The outputs from the neuroergonomic service can be accessed via one or more APIs. The outputs can be accessed in other ways besides APIs.

The neuroergonomic system 1100 includes a contextual server 1106 that runs a contextual service and outputs contextual data. In one example scenario, a user can permit events from activities on the laptop 1102(1) (e.g., the user's online browsing activities) to be transmitted via the network 1108 to the contextual server 1106. The contextual server 1106 can collect, parse, analyze, and format the received events into contextual data. In another implementation, events are sourced from the contextual server 1106 itself or from another server (e.g., a video game server, a movie streaming server, a videoconferencing server, etc.). The contextual data that is output from the contextual service on the contextual server 1106 can be accessed via one or more APIs. The outputs can be accessed in other ways besides APIs.

Although FIG. 11 shows the neuroergonomic service running on the neuroergonomic server 1104 and the contextual service running on the contextual server 1106 as cloud-based services, other configurations are possible. For example, the neuroergonomic service and/or the contextual service can run on a user computer, a laptop, or a smartphone, and incorporated into an end-user application.

FIG. 11 also shows two example device configurations 1110 of a user device, such as the laptop 1102(1), that includes a neuroergonomic application 1128 for receiving and presenting biosensing data and contextual data to users. The first device configuration 1110(1) represents an operating system (OS) centric configuration. The second device configuration 1110(2) represents a system on chip (SoC) configuration. The first device configuration 1110(1) can be organized into one or more applications 1112, an operating system 1114, and hardware 1116. The second device configuration 1110(2) can be organized into shared resources 1118, dedicated resources 1120, and an interface 1122 therebetween.

The device configurations 1110 can include a storage 1124 and a processor 1126. The device configurations 1110 can also include a neuroergonomic application 1128. For example, the neuroergonomic application 1128 can function similar to the neuroergonomic application 218, described above in connection with FIG. 2, and/or execute the neuroergonomic method 1000, described above in connection with FIG. 10.

As mentioned above, the second device configuration 1110(2) can be thought of as an SoC-type design. In such a case, functionality provided by the device can be integrated on a single SoC or multiple coupled SoCs. One or more processors 1126 can be configured to coordinate with shared resources 1118, such as storage 1124, etc., and/or one or more dedicated resources 1120, such as hardware blocks configured to perform certain specific functionality.

The term “device,” “computer,” or “computing device” as used herein can mean any type of device that has some amount of processing capability and/or storage capability. Processing capability can be provided by one or more hardware processors that can execute data in the form of computer-readable instructions to provide a functionality. The term “processor” as used herein can refer to central processing units (CPUs), graphical processing units (GPUs), controllers, microcontrollers, processor cores, or other types of processing devices. Data, such as computer-readable instructions and/or user-related data, can be stored on storage, such as storage that can be internal or external to the device. The storage can include any one or more of volatile or non-volatile memory, hard drives, flash storage devices, optical storage devices (e.g., CDs, DVDs etc.), and/or remote storage (e.g., cloud-based storage), among others. As used herein, the term “computer-readable medium” can include transitory propagating signals. In contrast, the term “computer-readable storage medium” excludes transitory propagating signals.

Generally, any of the functions described herein can be implemented using software, firmware, hardware (e.g., fixed-logic circuitry), or a combination of these implementations. The term “component” or “module” as used herein generally represents software, firmware, hardware, whole devices or networks, or a combination thereof. In the case of a software implementation, for instance, these may represent program code that performs specified tasks when executed on a processor (e.g., CPU or CPUs). The program code can be stored in one or more computer-readable memory devices, such as computer-readable storage media. The features and techniques of the component are platform-independent, meaning that they can be implemented on a variety of commercial computing platforms having a variety of processing configurations.

### CONCLUSION

The present concepts provide many advantages by presenting biosensing data in conjunction with contextual data. For example, the user can gain insights into the causes of physiological changes in people. This useful understanding can help people maintain good physical and mental wellbeing, and avoid negative and harmful conditions. Knowing the precise triggers of specific biosensing measurements can also help improve products, services, advertisements, meetings, workflow, etc., which can increase user satisfaction, boost workforce productivity, increase revenue, etc.

Communicating real-time data allows users to receive live data and immediately take corrective actions for the benefit of the users. For example, users can take a break from mentally intensive tasks that are negatively affecting the users. Communicating historical data about past sessions allows users to analyze past data and make improvements for future sessions.

Various examples are described above. Additional examples are described below. One example includes a system comprising a processor and a storage including instructions which, when executed by the processor, cause the processor to: receive biosensing measurements and biosensing metadata associated with the biosensing measurements, receive events including contextual metadata associated with the events, correlate the biosensing measurements with the events based on the biosensing metadata and the contextual metadata, generate a presentation of the biosensing measurements and the events, the presentation visually showing the correlation between the biosensing measurements and the events, and display the presentation to a user.

Another example can include any of the above and/or below examples where the biosensing measurements include sensor readings and cognitive state predictions.

Another example can include any of the above and/or below examples where the cognitive state predictions include one or more of: cognitive load levels, stress levels, affect states, and attention levels.

Another example can include any of the above and/or below examples where the biosensing measurements include a first set of measurements associated with the user and a second set of measurements associated with other users.

Another example can include any of the above and/or below examples where the instructions further cause the processor to calculate group metrics based on aggregates of the biosensing measurements for the user and the other users, and wherein the presentation includes the group metrics.

Another example includes a computer readable storage medium including instructions which, when executed by a processor, cause the processor to: receive biosensing data including sensor data and cognitive state data associated with a plurality of users and first timestamps, receive contextual data including event data associated with second timestamps, generate a presentation that includes the biosensing data and the contextual data in association with each other based on the first timestamps and the second timestamps, and display the presentation on a display screen.

Another example can include any of the above and/or below examples where the presentation shows a first portion of the biosensing data within a first time window and shows a second portion of the contextual data within a second time window, the first time window and the second time window being the same.

Another example can include any of the above and/or below examples where the instructions further cause the processor to receive a user input to adjust the second time window and automatically adjust the first time window based on the user input.

Another example includes A computer-implemented method, comprising receiving biosensing data, receiving contextual data, determining a correlation between the biosensing data and the contextual data, the correlation including a causal relationship, generating a presentation includes the biosensing data, the contextual data, and the correlation between the biosensing data and the contextual data, and displaying the presentation on a display screen.

Another example can include any of the above and/or below examples where the biosensing data includes a biosensing timeline, the contextual data includes a contextual timeline, and determining the correlation between the biosensing data and the contextual data includes aligning the biosensing timeline and the contextual timeline.

Another example can include any of the above and/or below examples where the biosensing data includes first identities of users, the contextual data includes second identifies of users, and determining the correlation between the biosensing data and the contextual data includes associating the first identities of users and the second identities of users.

Another example can include any of the above and/or below examples where the presentation includes a common time axis for the biosensing data and the contextual data.

Another example can include any of the above and/or below examples where the biosensing data includes one or more cognitive states associated with one or more users.

Another example can include any of the above and/or below examples where the one or more cognitive states include one or more of: cognitive load levels, stress levels, affect states, and attention levels.

Another example can include any of the above and/or below examples where the biosensing data includes sensor data associated with one or more users.

Another example can include any of the above and/or below examples where the sensor data includes one or more of: HRV, heart rates, EEG band power levels, body temperatures, respiration rates, perspiration rates, body motion measurements, or pupil sizes.

Another example can include any of the above and/or below examples where the contextual data includes events.

Another example can include any of the above and/or below examples where the events are associated with at least one of: a meeting, a video game, a movie, a song, a speech, or an advertisement.

Another example can include any of the above and/or below examples where the contextual data includes at least one of: texts, images, sounds, or videos.

Another example can include any of the above and/or below examples where the presentation is displayed in real-time.

1. A system, comprising:
a processor; and
a storage including instructions which, when executed by the processor, cause the processor to:
receive biosensing measurements and biosensing metadata associated with the biosensing measurements;
receive events including contextual metadata associated with the events;
correlate the biosensing measurements with the events based on the biosensing metadata and the contextual metadata;
generate a presentation of the biosensing measurements and the events, the presentation visually showing the correlation between the biosensing measurements and the events; and
display the presentation to a user.

2. The system of claim 1, wherein the biosensing measurements include sensor readings and cognitive state predictions.

3. The system of claim 2, wherein the cognitive state predictions include one or more of: cognitive load levels, stress levels, affect states, and attention levels.

4. The system of claim 1, wherein the biosensing measurements include a first set of measurements associated with the user and a second set of measurements associated with other users.

5. The system of claim 1, wherein the instructions further cause the processor to calculate group metrics based on aggregates of the biosensing measurements for the user and the other users, and wherein the presentation includes the group metrics.

6. A computer readable storage medium including instructions which, when executed by a processor, cause the processor to:
receive biosensing data including sensor data and cognitive state data associated with a plurality of users and first timestamps;
receive contextual data including event data associated with second timestamps;
generate a presentation that includes the biosensing data and the contextual data in association with each other based on the first timestamps and the second timestamps; and
display the presentation on a display screen.

7. The computer readable storage medium of claim 6, wherein the presentation shows a first portion of the biosensing data within a first time window and shows a second portion of the contextual data within a second time window, the first time window and the second time window being the same.

8. The computer readable storage medium of claim 7, wherein the instructions further cause the processor to:
receive a user input to adjust the second time window; and
automatically adjust the first time window based on the user input.

9. A computer-implemented method, comprising:
receiving biosensing data;
receiving contextual data;
determining a correlation between the biosensing data and the contextual data, the correlation including a causal relationship;
generating a presentation includes the biosensing data, the contextual data, and the correlation between the biosensing data and the contextual data; and
displaying the presentation on a display screen.

10. The computer-implemented method of claim 9, wherein:
the biosensing data includes a biosensing timeline;
the contextual data includes a contextual timeline; and
determining the correlation between the biosensing data and the contextual data includes aligning the biosensing timeline and the contextual timeline.

11. The computer-implemented method of claim 9, wherein:
the biosensing data includes first identities of users;
the contextual data includes second identifies of users; and
determining the correlation between the biosensing data and the contextual data includes associating the first identities of users and the second identities of users.

12. The computer-implemented method of claim 11, wherein the presentation includes a common time axis for the biosensing data and the contextual data.

13. The computer-implemented method of claim 9, wherein the biosensing data includes one or more cognitive states associated with one or more users.

14. The computer-implemented method of claim 13, wherein the one or more cognitive states include one or more of: cognitive load levels, stress levels, affect states, and attention levels.

15. The computer-implemented method of claim 9, wherein the biosensing data includes sensor data associated with one or more users.

16. The computer-implemented method of claim 15, wherein the sensor data includes one or more of: HRV, heart rates, EEG band power levels, body temperatures, respiration rates, perspiration rates, body motion measurements, or pupil sizes.

17. The computer-implemented method of claim 9, wherein the contextual data includes events.

18. The computer-implemented method of claim 17, wherein the events are associated with at least one of: a meeting, a video game, a movie, a song, a speech, or an advertisement.

19. The computer-implemented method of claim 9, wherein the contextual data includes at least one of: texts, images, sounds, or videos.

20. The computer-implemented method of claim 9, wherein the presentation is displayed in real-time.
